(19) United States
(12) Patent Application Publication
(10) Pub. No.: US 2022/0354579 A1
(43) Pub. Date: Nov. 10, 2022

(54) SYSTEMS AND METHODS FOR PLANNING AND SIMULATION OF MINIMALLY INVASIVE THERAPY

(71) Applicant: SYNAPTIVE MEDICAL INC., Toronto (CA)

(72) Inventors: Kelly Noel DYER, Toronto (CA); Fergal KERINS, Toronto (CA); Alicia MCNEELY, Toronto (CA)

(21) Appl. No.: 17/302,658

(22) Filed: May 10, 2021

(51) Int. Cl. A61B 34/10; A61B 34/20; G02B 27/01; A61B 34/00

(52) U.S. Cl. CPC A61B 34/10; A61B 34/20; G02B 27/017; A61B 34/76; A61B 2034/105; A61B 2034/107; A61B 2034/102; A61B 2034/2046

(57) ABSTRACT

A system and method is provided for planning a surgical procedure. The system includes a model of an anatomical region including simulated bone, a tracked tool, a tracking system, a display device, and a computer system configured to receive information from the tracking system, generate and display the model and the tracked tool, and store the tracking system information in a computer readable memory that is portable to a surgical navigation system. The method includes acquiring volumetric data of a patient anatomical region, extracting, providing and registering a three-dimensional model with the volumetric data, using a tracked tool to perform a simulated surgical procedure including removing and replacing a bone section on the model, viewing the model and the tracked tool on a display device, recording the procedure with a tracking system, storing the procedure in a computer readable memory, and porting the procedure into a surgical navigation system.

### FIELD

The present disclosure relates to surgical planning and methods for minimally invasive therapy and image guided medical procedures.

### BACKGROUND

In the field of medicine, invasive surgery is frequently assisted by imaging and image guidance. In neurosurgery, for example, brain tumors are typically excised through an open craniotomy approach guided by imaging. Imaging data may consist of Computed X-ray tomography (CT), Magnetic Resonance Imaging (MRI), Ultrasound (US), and Positron Emission Tomography (PET), and may be obtained pre-operatively for use in surgical navigation. Tracking of instruments relative to the patient and the associated imaging data is also often achieved by way of external hardware systems such as mechanical arms, or radiofrequency or optical tracking devices. As a set, these devices are commonly referred to as surgical navigation systems.

Since image-guided surgical procedures are complex in nature, the surgical staff must often resort to performing a simulated rehearsal of the entire procedure. Unfortunately, the tools and models that are currently available for such simulated rehearsal and training exercises typically fail to provide a surgical planning system. Thus, there is a need for a system and method to provide tools to the surgeon that assist in planning a surgery.

### SUMMARY

A further understanding of the functional and advantageous aspects of the invention can be realized by reference to the following detailed description and drawings.

An object of the present disclosure is to provide planning of a surgical procedure. The system includes a model of an anatomical region including simulated bone, a tracked tool, a tracking system, a display device, and a computer system configured to receive information from the tracking system, generate and display the model and the tracked tool, and store the tracking system information in a computer readable memory that is portable to a surgical navigation system. The method includes acquiring volumetric data of a patient anatomical region, extracting, providing and registering a three-dimensional model with the volumetric data, using a tracked tool to perform a simulated surgical procedure including removing and replacing a bone section on the model, viewing the model and the tracked tool on a display device, recording the procedure with a tracking system, storing the procedure in a computer readable memory, and porting the procedure into a surgical navigation system.

Thus by one broad aspect of the present disclosure a planning system for a surgical procedure by a practitioner on a patient anatomical region is provided, comprising a model of the patient anatomical region including simulated bone, a tracked tool for interacting with the model, a tracking system configured to track the tracked tool location and orientation relative to the model, a display device for viewing the model and the tracked tool, a computer system electronically connected to the tracking system and the display device, the computer system being configured to receive information from the tracking system indicating the tracked tool location and orientation, generate and display the model and the tracked tool as the tracked tool performs the surgical procedure on the model, and store the tracking system information in a computer readable memory that can be ported to a surgical navigation system.

By a further broad aspect of the present disclosure, a method of surgical planning is provided, comprising acquiring a volumetric data of a patient anatomical region using a medical imaging methodology, extracting a three-dimensional model of the patient anatomical region from the volumetric data, providing the model, registering the model with the volumetric data, using a tracked tool to perform a simulated surgical procedure on the model, wherein the surgical procedure comprises removing a bone section from the model and reassembling the model with an implant, viewing the model and the tracked tool on a display device, recording the simulated surgical procedure with a tracking system, storing the surgical procedure in a computer readable memory, and porting the surgical procedure into a surgical navigation system.

### BRIEF DESCRIPTION OF THE DRAWINGS

Embodiments disclosed herein will be more fully understood from the following detailed description thereof taken in connection with the accompanying drawings, which form a part of this application, and in which:

FIG. 1 shows an exemplary operating room setup for a minimally invasive access port-based medical procedure.

FIG. 2 illustrates the insertion of an access port into a human brain during a medical procedure.

FIG. 3 is a systems diagram illustrating a planning system used for a medical procedure.

FIG. 4 is a flow chart illustrating steps in the planning system.

FIG. 5A-G are exemplary screenshots illustrating the planning software at each step in FIG. 4.

FIG. 6 is a block diagram illustrating a control and processing system that may be used in the planning system shown in FIG. 3.

FIG. 7 is a block diagram showing system components and inputs for planning and scoring surgical paths as disclosed herein.

FIG. 8 is a block diagram showing system components and inputs for navigation along the surgical paths produced by the planning system of FIG. 7.

FIG. 9 is a schematic diagram showing the elements of an embodiment of the invention.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

The systems and methods described herein are useful in the field of neurosurgery, including oncological care, neurodegenerative disease, stroke, brain trauma and orthopedic surgery; however, persons of skill will appreciate the ability to extend these concepts to other conditions or fields of medicine. It should be noted that the surgical process is applicable to surgical procedures for brain, spine, knee and any other region of the body that will benefit from the use of an access port or small orifice to access the interior of the human body, and to surgical procedures involving bone.

Various apparatuses or processes will be described below to provide examples of embodiments of the surgical planning method and system disclosed herein. No embodiment described below limits any claimed embodiment and any claimed embodiments may cover processes or apparatuses that differ from those described below. The claimed embodiments are not limited to apparatuses or processes having all of the features of any one apparatus or process described below or to features common to multiple or all of the apparatuses or processes described below. It is possible that an apparatus or process described below is not an embodiment of any claimed invention.

Furthermore, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein.

Unless defined otherwise, all technical and scientific terms used herein are intended to have the same meaning as commonly understood to one of ordinary skill in the art. Unless otherwise indicated, such as through context, as used herein, the following terms are intended to have the following meanings:

As used herein, the phrase “access corridor” or “access port” refers to a cannula, conduit, sheath, port, tube, or other structure that is insertable into a subject, in order to provide access to internal tissue, organs, or other biological substances. In some embodiments, an access port may directly expose internal tissue, for example, via an opening or aperture at a distal end thereof, and/or via an opening or aperture at an intermediate location along a length thereof. In other embodiments, an access port may provide indirect access, via one or more surfaces that are transparent, or partially transparent, to one or more forms of energy or radiation, such as, but not limited to, electromagnetic waves and acoustic waves.

As used herein, the phrase “intraoperative” refers to an action, process, method, event or step that occurs or is carried out during at least a portion of a medical procedure. Intraoperative, as defined herein, is not limited to surgical procedures, and may refer to other types of medical procedures, such as diagnostic and therapeutic procedures.

As used herein, the phrase “pre-operative” refers to an action, process, method, event or step that occurs or is carried out prior to a related medical procedure and may refer to procedures or steps carried out in preparation for a surgical, diagnostic or therapeutic procedure.

As used herein, the phrase “navigation system” refers to a system that assists in surgery by providing previously acquired imaging information during surgery to visualize tissue morphology and locate target areas. Navigation systems may also be used to track surgical instruments and their location within the tissue during surgery, typically incorporating information from previously acquired imaging data.

As used herein, the phrase “positional tracking system” refers to a computer-implemented system that tracks the position of surgical instruments during surgery. A positional tracking system may be incorporated in a navigation system or may function independently of a navigation system. Where embodiments of the present disclosure refer to a navigation system, an independent positional tracking system may be alternately used.

Traditionally, brain tumors and intracranial hemorrhages (ICH) are treated by removing most of the top half of the patient's skull and resecting healthy white matter to get to the tumor or ICH of interest. This approach has the following obvious disadvantages of: Permanent removal of healthy white matter; Increased trauma to the brain via de-pressurization after removal of a large portion of the skull; and Long recovery time due to large cranial trauma

The neurosurgeon is typically guided in these procedures using a navigation system that displays the position of surgical tools overlaid on pre-surgical MR or CT images in real-time. In these procedures, one or more targets and a surgical path are defined. An ideal surgical path will be determined by the surgeon before the surgery but is not encoded or reflected by the navigation system.

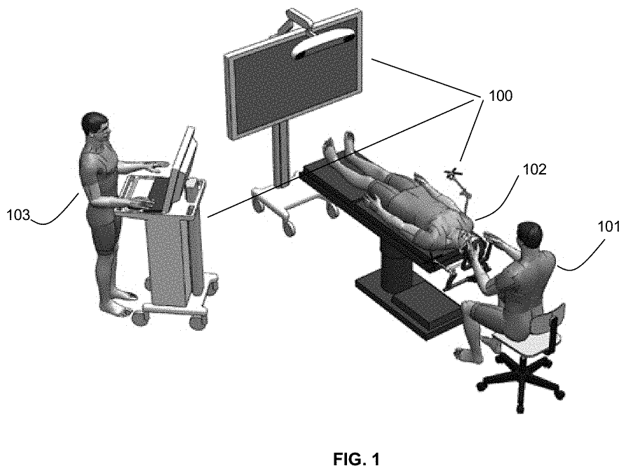

FIG. 1 shows an exemplary navigation system to support minimally invasive access port-based surgery. In FIG. 1, neurosurgeon 101 conducts a minimally invasive port-based surgery on a patient 102 in an operating room (OR) environment. A navigation system 100 comprising an equipment tower, tracking system, displays and tracked instruments assist the surgeon 101 during his procedure. An operator 103 is also present to operate, control and provide assistance for the navigation system 100.

A new approach to resection of brain tumors and ICHs is the use of a small port to access the tumor or ICH. The port is typically a hollow tube inserted into the brain for the purpose of minimally-invasive neurosurgery. The port is inserted via a very small burr hole craniotomy into a sulcus of the brain. Because it follows a sulcus, the port compromises less white matter. Resection of the tumor is conducted via instruments inserted into the port.

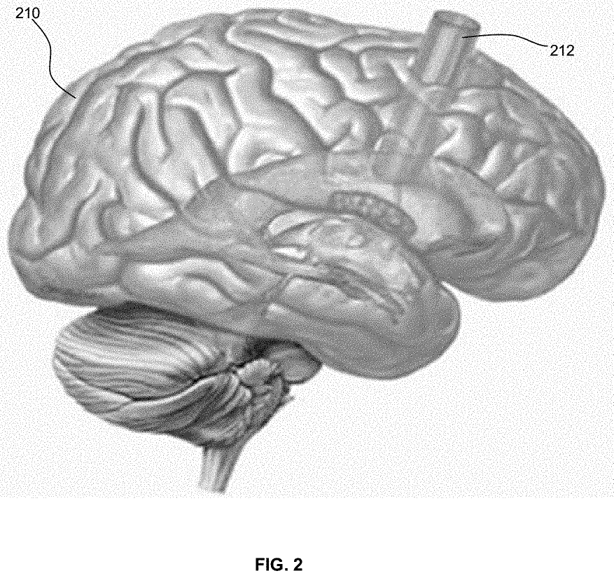

As an example, FIG. 2 shows an access port 212 inserted into a human brain 210, providing access to internal brain tissue. Surgical tools and instruments may then be inserted within the lumen of the access port in order to perform surgical, diagnostic or therapeutic procedures, such as resecting tumors as necessary. This approach allows a surgeon, or robotic surgical system, to perform a surgical procedure involving tumor resection in which the residual tumor remaining after is minimized, while also minimizing the trauma to the intact white and grey matter of the brain. In such procedures, trauma may occur, for example, due to contact with the access port, stress to the brain matter, unintentional impact with surgical devices, and/or accidental resection of healthy tissue.

Performing the port-based surgery is conducted via image guidance similar to the traditional image-guided surgery described above. In the case of port-based surgery, the engagement point (or starting point) for insertion of the port is carefully selected to minimize trauma to eloquent function of the brain. For this reason, both the navigation system and the planning system require the concept of a trajectory to be planned, encoded, and executed.

To make the procedure least disruptive to eloquent function, it is helpful to visualize tracts of oriented diffusion in the brain. These tracts can be generated from DTI (diffusion tensor imaging) image sequences. A plan that avoids damage to these tracts (by avoiding intersection of tracts with the port) is preferred.

A craniotomy is performed at the engagement point for insertion of the port. The section of the skull, or bone flap, that is removed in the craniotomy may be replaced in the skull at the end of the surgery. Replacement of the bone flap may be impeded by swelling of the underlying soft tissue and surgical planning can assist in anticipating and preparing for such complications. In other types of surgeries, a deformation of the cranium or a traumatic injury may require surgical correction wherein bone is removed and repositioned. In a further example, a deformation of the cranium or a traumatic injury may require surgical correction, in which bone may be removed and a bone graft or an implant provided to replace or correct bone structure. The present disclosure provides planning systems and methods in which to test and rehearse a surgical procedure including surgical correction of a bone deformation or injury. A planning system and model in which to test and rehearse how a bone flap, bone graft or implant will fit and should be inserted provides an opportunity to optimize the surgical procedure and determine steps required for a successful outcome. Such steps can be recorded by the planning system, which can then be implemented in a surgical plan through the navigation system.

### Systems Overview

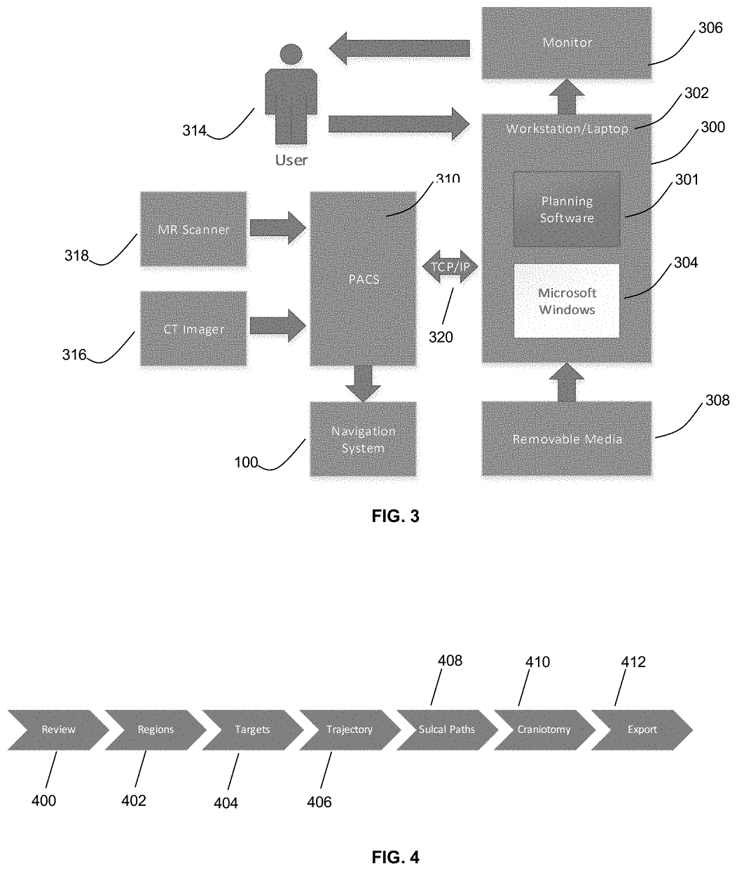

FIG. 3 illustrates an overall environment of a medical planning system 300 within which the planning software operates. In FIG. 3, planning software 301 is typically installed on a computer 302. Computer 302 may be a workstation computer, laptop, tablet, mobile device or a wearable computer device. The preferred operating system 304 of computer 302 is Windows, but other operating systems such as Mac, Linux, QNX, iOS, Android, BlackBerry may also be envisioned by persons skilled in the art.

Computer 302 is connected to one or more display monitor(s) 306, where the output data is shown to user 314. User 314 may be a nurse, operator 103, neurosurgeon 101, or a user of planning software 301 on computer 302. Computer 302 may also receive input from removable media 308. Removable media 308 may include a CD-ROM, Blu-Ray disk, Universal Serial Bus (USB) memory stick, external hard drive, or other memory storage devices.

Planning software 301 is envisioned to make the planning of port-based procedures more effective and efficient than current practice Planning software 301 on computer 302 also interfaces with a picture archiving and communication system (PACS) 310 typically over a TCP/IP 320 network communication interface. PACS 310 may receive input from a Magnetic Resonance (MR) Scanner 318 in the form of MR Images, or with a Computed Tomography (CT) Imager 316 in the form of Computerized Axial Tomography (CAT) scans. As seen in FIG. 3, PACS 310 interfaces with two modalities (MR and CT), but other and/or additional modalities such as Optical Coherence Tomography (OCT), Positron Emission Tomography (PE)T, and Ultrasound (US) may exist. Finally, PACS 310 also interfaces with navigation system 100 where the output of planning software 301 may be considered as the initial input to navigation system 100 to be used in a port-based medical procedure.

In addition to port-based procedures, planning software 301 may also be used to support planning of functional deep brain stimulation (DBS) procedures, neural biopsy procedures, catheter/shunt placement, and bone reconstruction such as craniofacial bone reconstruction. In all these cases, the procedure involves a bone incision.

While one example of a planning system 300 is provided that may be used with aspects of the present application, any suitable planning or navigation system may be used, such as a planning system using magnetic tracking instead of infrared cameras, and/or active tracking markers.

According to one aspect of the present application, one purpose of the planning system 300 is to provide tools to the neurosurgeon that will lead to the most informed, least damaging neurosurgical operations. In addition to removal of brain tumors and intracranial hemorrhages (ICH), the planning system 300 can also be applied to a brain biopsy, a functional/deep brain stimulation (DBS), a catheter/shunt placement procedure, open craniotomies, endonasal/skull-based/ENT, spine procedures, and other parts of the body such as knee, etc. While several examples have been provided, aspects of the present disclosure may be applied to any suitable medical procedure.

### Software Flow

FIG. 4 is a flow chart illustrating the steps taken in planning software to assist in the planning of a surgical procedure. As described here, the surgical procedure may be a tumor resection. However, planning software can be used similarly for other types of brain surgical procedures or surgical procedures involving removal and replacement of bone tissue. As seen in FIG. 4, planning software 301 (as shown in FIG. 3) consists of 7 steps which includes the following:

### Review Phase

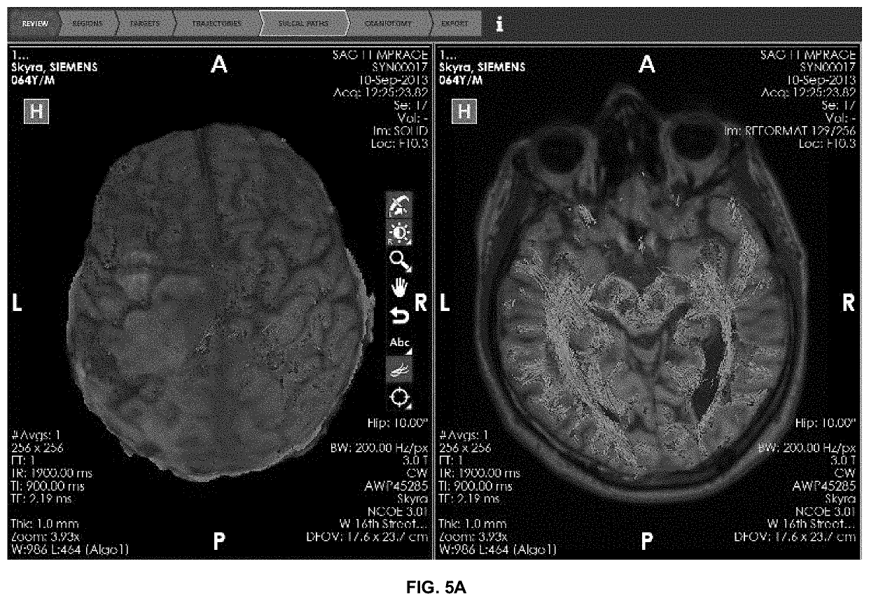

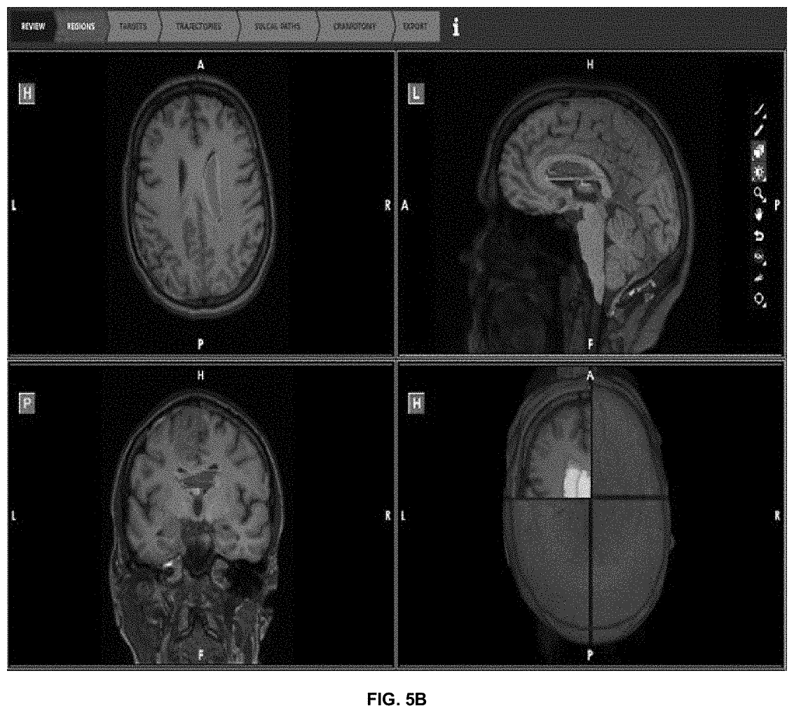

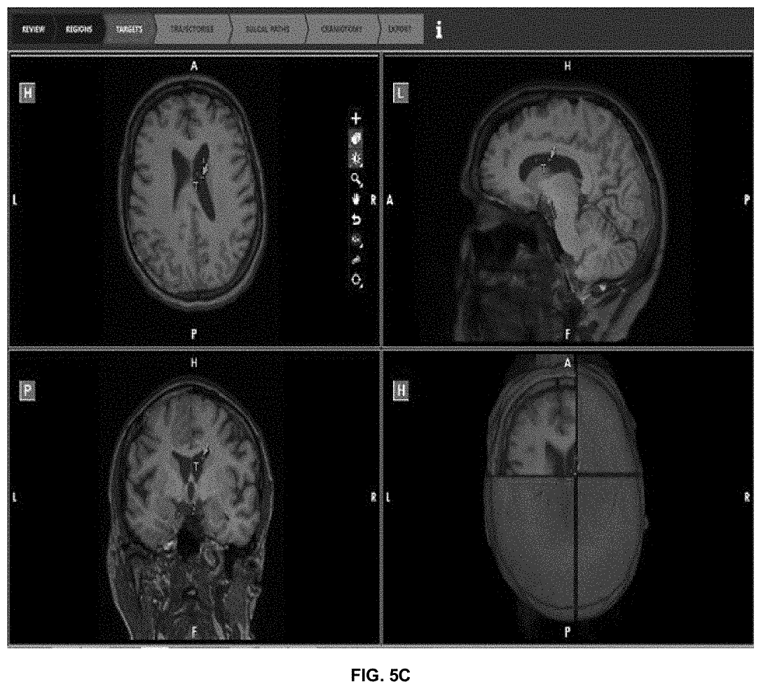

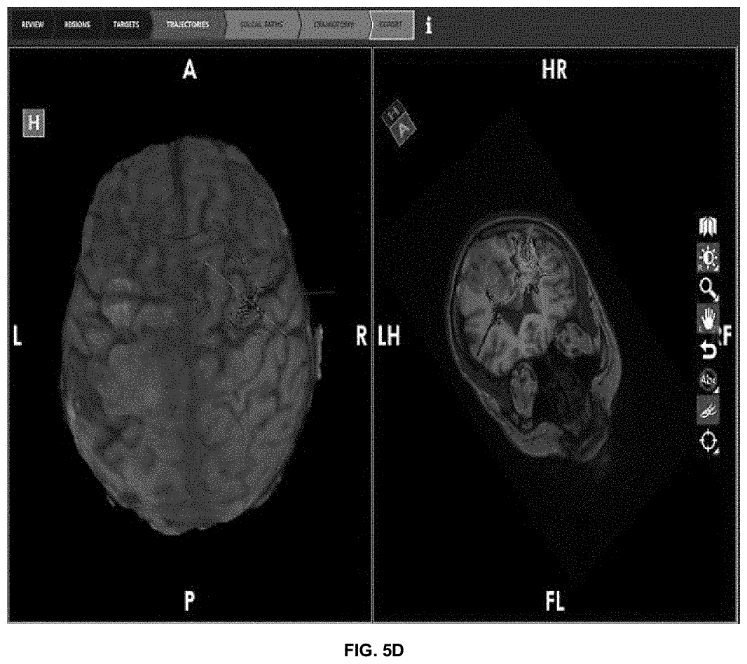

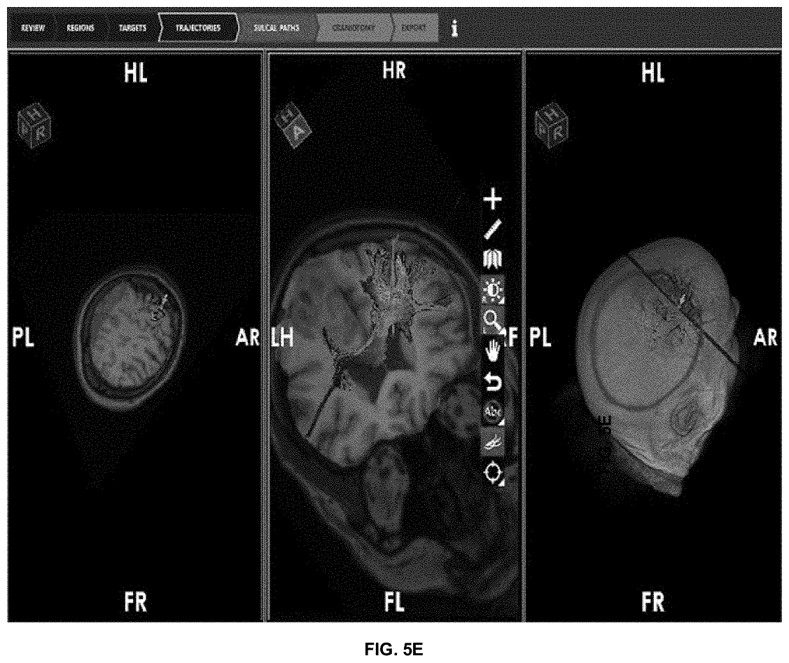

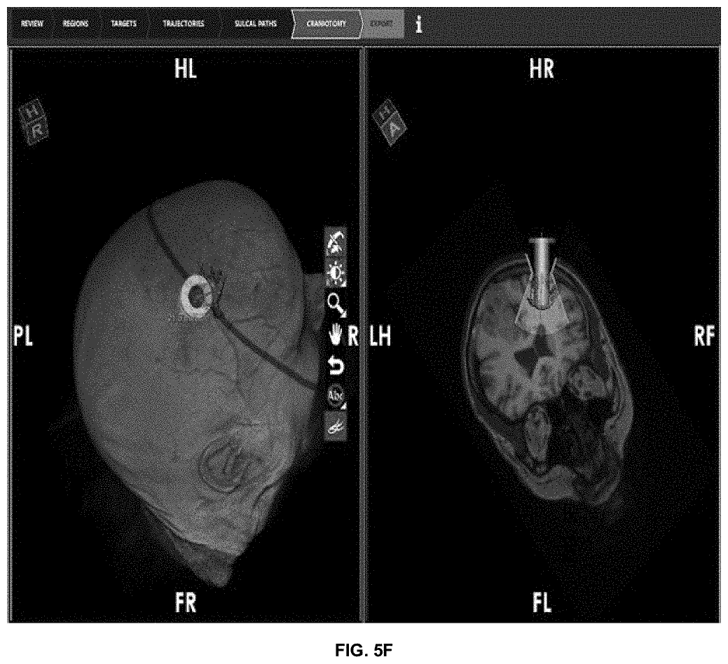

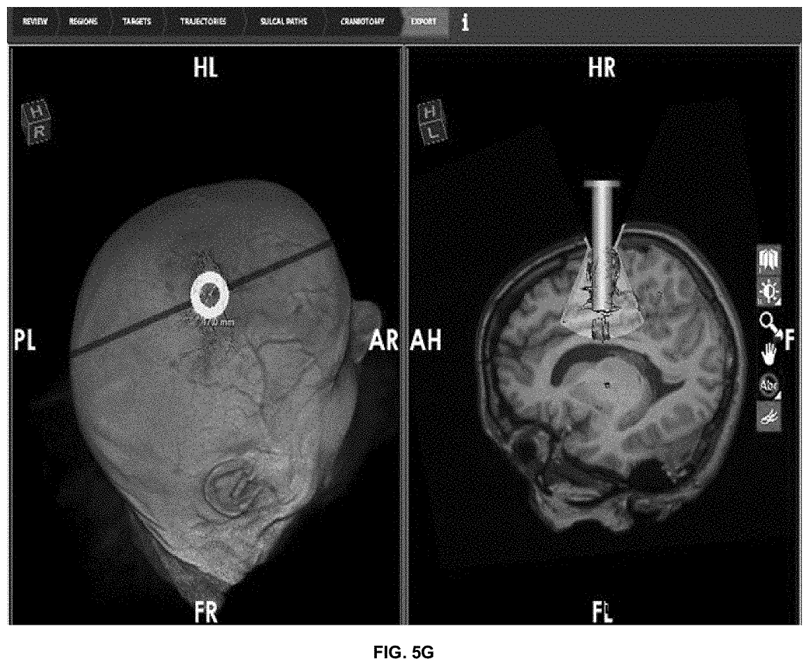

The first step is the review phase (step 400). The review phase (step 400) provides a user interface model for accepting brain mask, diffusion, and tractography data available in the study. A screen shot of review phase 400 can be seen in FIG. 5A.

### Regions Phase

The next step is the regions phase (step 402) which allows the user to define regions of interest in clinical images to aid in development of a surgical plan. The regions phase (step 402) provides a user interface model for annotating volumes of interest within the primary image series, as well as co-registered Fractional Anisotropy (FA) and Apparent Diffusion Coefficient (ADC) image series, if they are available. The volumes of interest may aid in visualizing and placing candidate target locations within subsequent phases. A screen shot of regions phase 402 can be seen in FIG. 5B.

### Targets Phase

Thereafter, the process moves to the targets phase (step 404). The targets phase (step 404) provides a user interface model for visualizing and placing target locations in and around tumor locations. A target location is the endpoint of a surgical cannulation using a port. A screen shot of the targets phase 404 can be seen in FIG. 5C.

### Trajectory Phase

The next step is the trajectory phase (step 406) which identifies a target and engagement points to define intended trajectories to approach pathology regions. The trajectory phase (step 406) provides a user interface model for placing points of entry from a location on the surface of the brain to a target location in order to form a trajectory for a surgical cannulation using a port. This phase also provides a scorecard user interface model to provide comparisons between trajectory characteristics to this point in the workflow. A screen shot of trajectory phase 406 can be seen in FIG. 5D.

### Sulcal Paths Phase

Following the trajectory phase (406) is the sulcal paths phase (408). The sulcal paths phase (408) provides a user interface model for visualizing and placing waypoints for a surgical cannulation along a trajectory from engagement point to target, typically along a sulcal path. The sulcal paths phase (408) allows the user to segment the path through the sulcus and evaluate how this path might differ from the theoretical direct trajectory. A screen shot of this phase can be seen in FIG. 5E.

### Craniotomy Phase

The next step is the craniotomy phase (step 410). This phase allows the user to define the preferred dimension for craniotomy using regions of interest, target and engagement points. In the craniotomy phase (step 410), a user interface model for estimating the location and size of the craniotomy required to support a trajectory for surgical cannulation using a port is provided. A screen shot of this phase can be seen in FIG. 5F.

### Export Phase

Finally, the last phase is the export phase (step 412). The export phase (step 412) provides a user interface model for reviewing and exporting one or more trajectory plans as an image series to a PACS for subsequent use in suitable surgical navigation systems. A screen shot of this phase can be seen in FIG. 5G.

The latter four phases (steps 406, 408, 410, 412) may provide the user with a scorecard user interface model to provide comparisons between trajectory characteristics to this point in the workflow.

In a further embodiment, parallel steps for bone reconstruction planning can be considered. The steps for bone reconstruction planning include:
review phase provides a user interface model for accepting brain mask, diffusion and tractography data, and/or bone and soft tissue image data,
regions phase provides a user interface model to annotate volumes of interest in clinical images,
targets phase to provide annotation of target bone shape or skull volume,
trajectory phase identifies placement of implant, provides user interface model for marking points of insertion of bone or implant,
craniotomy phase to outline bone to be removed, and
export phase provides a user interface model for review and export plans as an image series to a PACS for subsequent use in surgical navigation systems

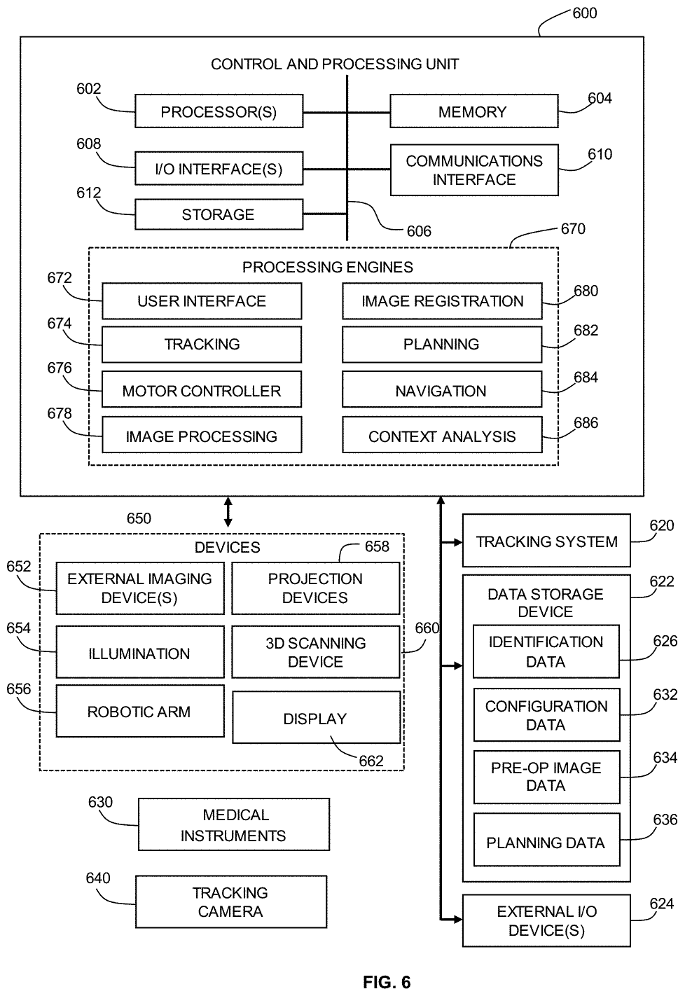

Referring to FIG. 6, a block diagram is shown illustrating a control and processing system 600 that may be used with the medical planning software 301 shown in FIG. 3. The control and processing system allows a practitioner to rehearse and test a surgical procedure.

As shown in FIG. 6, in one example, control and processing system 600 may include one or more processors 602, a memory 604, a system bus 606, one or more input/output interfaces 608, a communications interface 610, and storage device 612. Control and processing system 600 may be interfaced with other external devices, such as tracking system 620, data storage 622, and external user input and output devices 624, which may include, for example, one or more of a display, keyboard, mouse, sensors attached to medical equipment, foot pedal, and microphone and speaker. Data storage 622 may be any suitable data storage device, such as a local or remote computing device (e.g. a computer, hard drive, digital media device, or server) having a database stored thereon. In the example shown in FIG. 6, data storage device 622 includes identification data 626 for identifying one or more medical instruments 630 and configuration data 632 that associates customized configuration parameters with one or more medical instruments 630. Data storage device 622 may also include preoperative image data 634 and/or medical procedure planning data 636. Although data storage device 622 is shown as a single device in FIG. 6, it will be understood that in other embodiments, data storage device 622 may be provided as multiple storage devices.

Medical instruments 630 are identifiable by control and processing unit 600. Medical instruments 630 may be connected to and controlled by control and processing unit 600, or medical instruments 630 may be operated or otherwise employed independent of control and processing unit 600. Tracking system 620 may be employed to track one or more of medical instruments 630 and spatially register the one or more tracked medical instruments to an intraoperative reference frame. For example, medical instruments 630 may include tracking markers such as tracking spheres that may be recognizable by a tracking camera 640. In one example, the tracking camera 640 may be an infrared (IR) tracking camera. In another example, a sheath placed over a medical instrument 630 may be connected to and controlled by control and processing unit 600.

Control and processing unit 600 may also interface with a number of configurable devices, and may intraoperatively reconfigure one or more of such devices based on configuration parameters obtained from configuration data 632. Examples of devices 650, as shown in FIG. 6, include one or more external imaging devices 652, one or more illumination devices 654, an automated or robotic arm 656, one or more projection devices 658, one or more 3D scanning devices 660 (such as 3D scanners or structured light scanners), and one or more displays 662. Examples of external imaging devices 652 include optical coherence tomography (OCT), computed tomography (CT), magnetic resonance imaging (MRI), positron emission tomography (PET) and ultrasound (US) imaging devices.

Exemplary aspects of the disclosure can be implemented via processor(s) 602 and/or memory 604. For example, the functionalities described herein can be partially implemented via hardware logic in processor 602 and partially using the instructions stored in memory 604, as one or more processing modules or engines 670. Example processing modules include, but are not limited to, user interface engine 672, tracking module 674, motor controller 676, image processing engine 678, image registration engine 680, procedure planning engine 682, navigation engine 684, and context analysis module 686. While the example processing modules are shown separately in FIG. 6, in one example the processing modules 670 may be stored in the memory 604 and the processing modules may be collectively referred to as processing modules 670.

It is to be understood that the system is not intended to be limited to the components shown in FIG. 6. One or more components of the control and processing system 600 may be provided as an external component or device. In one example, navigation module 684 may be provided as an external navigation system that is integrated with control and processing system 600.

Some embodiments may be implemented using processor 602 without additional instructions stored in memory 604. Some embodiments may be implemented using the instructions stored in memory 604 for execution by one or more general purpose microprocessors. Thus, the disclosure is not limited to a specific configuration of hardware and/or software.

While some embodiments can be implemented in fully functioning computers and computer systems, various embodiments are capable of being distributed as a computing product in a variety of forms and are capable of being applied regardless of the particular type of machine or computer readable media used to actually effect the distribution.

### Surgical Planning Tool

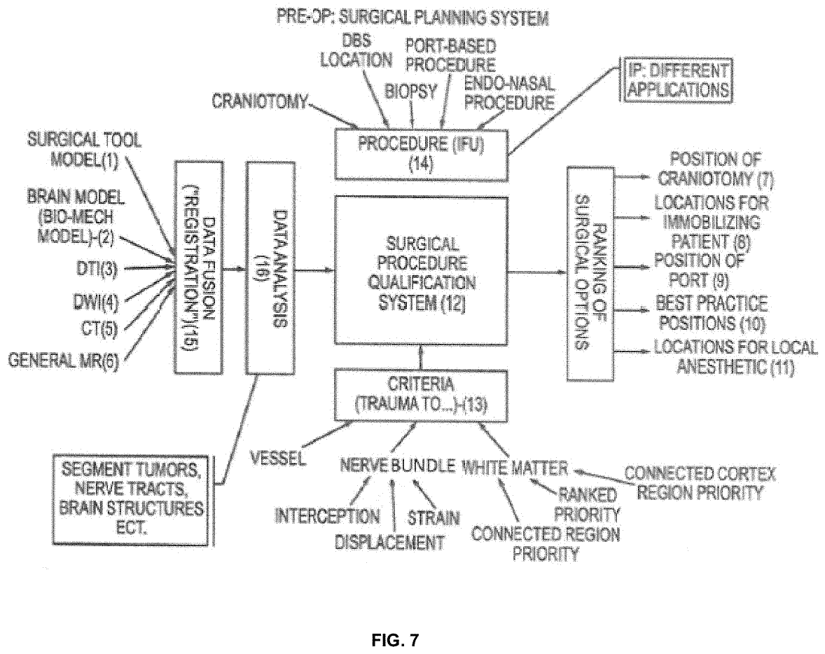

FIG. 7 shows an embodiment of the present system and method, for use as a multi-modal surgical planning tool. The system and method can be used as a surgical planning tool in the pre-operative stage. Persons of skill will appreciate that the surgical planning steps depicted in FIG. 7 may also be repeated intra-operatively to further refine the surgical approach, such that the terms surgical planning and intra-operative navigation may be used interchangeably.

In some embodiments, the systems and methods may include data inputs including but not limited to tracked surgical instruments or surgical tool models (1) and sensors, and bio-mechanical models (2) of tissues and organs. The data input(s) can include images from various modalities including MRI (6), CT (5) or PET, as well as data from tracking or navigation systems, including tracked surgical devices, such as scissors, ablation devices, suction cutters, bi-polars, tracked access port devices and automated guidance external imaging systems. In some embodiments, particular surgical procedures (14) and clinical criteria (13), selected for example on a patient by patient basis, can be utilized as additional input(s) to assess optimal surgical plans. Imaging data may be acquired by comparing various images of the patient's tissue and organs, including co-registered data between DWI (diffusion weighted imaging) (4), DTI (diffusion tensor imaging) (3), and other imaging contrast sequences and modalities. In an embodiment where the present invention is used in an intra-operative setting, to set or update a surgical path, data inputs may include examples from the above imaging, acquired through sensors, as is further disclosed herein. Sensor(s) may include means for accurately and robustly tracking surgical tools, including optical or electromagnetic intra-operative tracking components, and other means of registration (15) of the intra-operative data sets to the pre-operative dataset. Registration methods can include, for example, any or a combination of the following: image intensity matching based on similarity metrics such as sum of squared intensity differences and mutual information, computed over neighborhoods or regions; image feature based registration such as edge matching; fiducial or anatomical feature based matching of common points defined in multiple image modalities or coordinate spaces (such as a tracking system's coordinate space and an MR image's coordinate space); surface matching techniques such as surface mesh matching.

Persons of skill will appreciate that the sensor(s) can also include planning, navigation and modeling components, contextual interfaces, intraoperative imaging devices, devices for bi-polar suction, tissue ablation and tissue cutting with attached imaging, tracking technologies, including external and internal tool tracking (light deflection, capacitive, strain gauge), automated guidance external imaging systems, semi-automated external positioning arms with turrets, internal semi-automated manipulators, multiple beam delivery systems, databases with adaptive learning networks, imaging and spatially linked pathology systems, imaging devices which respond to the context they are used in, as well as user interfaces which respond to the context and environment they are used in.

Inputs and sensor(s) can also include keyboards, touch screens, pointers or tools that act as pointing devices, mice or gesture control components.

Data inputs further include a patient anatomical model (2), for example a brain model including a skull upon which to perform a pre-operative procedure.

Surfaces can be manually outlined or automatically segmented from image data. Similarly, surfaces can be determined from the physical patient or patient model by outlining with a tracked pointer tool or through a surface scanning technique (such as a laser rangefinder, structured light system or stereo camera). All matching and registration methods can be performed on a sub-region of an image or patient volume (such as what is visualized through the port), to focus on a specific region of interest. Registration can be performed on multiple sub-regions jointly or independently and an interpolated registration can be inferred between these independent regions. Once the images are registered they form an input to a data analysis module (16).

In some embodiments, the processor(s) may include planning module(s) (12) that analyze input(s) from criteria metrics (13) and data analysis (16) to define surgical procedures (14). These may include open craniotomies, DBS stimulator locations, biopsy sites, port-based or minimal corridor approaches and endo-nasal based approaches based on a variety of input(s) and rule-based calculations. In further embodiments, the processor(s) may include navigation module(s) that analyze input(s) to provide visualization and other outputs during procedures, such as tool tracking, and contextual information.

In other embodiments, the processor(s) may segment tissue structures such as tumors, nerves and nerve tracts, brain structures, such as ventricles, sulci, cortex, white matter, major white matter bundles, vasculature such as arteries and veins, and boney structures such as skull and brain stem, for planning and navigation purposes.

Output(s) can include 2D and 3D composite images, used for guidance, including tissue extraction guidance and guidance for devices including DBS probes and biopsy probe. Persons of skill will appreciate that output device(s), including monitors or laser pointers can also be included in the systems and methods described herein to provide users with feedback on the processes of the system.

Visualization output(s) can include contextual volume imaging; point source imaging which involves imaging only the regions of interest that are important at that point of the surgical procedure; imaging to check positioning before instrument insertion or removal, imaging to update tissue maps after resection, as well as imaging to resect maximal tumor while limiting damage to healthy or recoverable tissue. In addition, the use of common contrast mechanisms between imaging modalities used in the systems and methods described herein may allow the processor(s) to generate accurate registration between modalities, and meaningful volumetric imaging updates during procedures.

Output(s) can also include path planning or correction data for a surgical approach by way of feature detection, positions for procedures such as craniotomies, locations for pinning and immobilization of patients.

Output(s) can also include data on selection of surgical approach, for instance trans-sulcal approaches to avoid vessels and fiber bundles. For example, the output(s) can also include sulci based approach paths to minimize white matter and grey matter insertion damage. Further output(s) can include parametric curves or volumes to define or facilitate a time evolution of data such as the chosen paths, tissue deformations, time animation of data sets with time components (e.g. Doppler US or fMRI), or arbitrary combinations of such data.

### Anatomical Models

The system according to the present invention employs a model of a patient anatomical region that replicates the patient anatomical features. The model may comprise a physical model or may be an augmented reality model, having a combination of physical and virtual components. For example, a model may include a physical skull component suitable for simulating the surgical procedure of a craniotomy, and a brain and brain tumor target as computer-generated virtual components of the model. In another example, a skull may be a physical component of the model overlaid with virtual soft tissue, providing a mixed reality model wherein a craniofacial reconstruction can be planned. A combination of physical and virtual components is used in the planning system to provide an optimal simulation of a surgical procedure and patient anatomical features. The model is generated based on pre-operative medical imaging of the specific patient features. Thus, the model is generated to provide the patient-specific anatomical features for surgical planning and rehearsal.

To acquire a patient-specific model, patient image data is acquired, such as volumetric data obtained from low-dose CT or MR. Patient-specific anatomical features can also be acquired using sensors such as pointer tools to touch or trace a feature. Patient-specific details, such as shape and size of the cranium, bone defects, lack of bone structure, locations of seams of the skull are recorded and stored as part of the pre-operative image acquisition. The image data is registered and used to extract a 3D model of the patient anatomy, for example the cranium. The 3D model may be a computer-generated virtual model, a physical 3D model built using a process such as 3D printing or a mixed virtual and physical model i.e. an augmented physical model. In the model, simulated bone may comprise of hydroxyapatite (hydroxylated calcium phosphate) or other similar materials that is known in the art. In another example, simulated bone and soft tissue may comprise virtual tissue grafts, for example a submental island flap, the donor tissue planned and visualized using a patient model from pre-operative imaging as described herein.

The medical procedure may involve perforating, drilling, boring, punching, piercing, or any other suitable methods, as necessary for an endo-nasal, port-based, or traditional craniotomy approach. The simulated bone may be excised and for surgeries requiring replacement of the excised bone, the simulated bone or implant may be flexed to fit the excision site to reassemble the skull model. In a craniotomy requiring excision across seams of the skull, the pre-surgical planning system allows a neurosurgeon to test alternate approaches and determine the optimal procedure for cutting and replacing the skull segment to reassemble the skull.

### General Planning Method for Any Part of a Patient's Body

Disclosed herein is a planning method executed on a computer for planning a surgical procedure including bone incision. The method includes acquiring pre-operative images of a portion of the patient's body to be operated on using at least one imaging modality configured for acquiring a 3D image data set or volume and storing the 3D image data set or volume in a storage medium. It will be understood that more than one imaging modality may be used, in particular where the anatomical part of the patient to be operated on would best be suited to a certain type or combination of imaging modality. An image and/or a model of a 3D volume is produced from the 3D image data set. The model may be produced by 3D printing. The image of the 3D volume is stored in the storage medium. Once the location of the one or more targets has been identified, their location(s) may be adjusted and/or confirmed on 2D planar estimates or projections of the data, referred to as “reformats”. This technique visualizes representations of one or more 2D planes through the 3D space containing the image data. Such planes are often orthogonal, and often shown in canonical (axial, coronal, sagittal) directions as a “multiplanar reconstruction” or “MPR”. Other variants exist, such as “radial stacking” where one or more planes are shown through a common axis about which they all rotate. However it will be appreciated that any configuration of planes, containing image data from a single source or fusions of multiple sources may be used. Where 3D data exists (such as from an MRI, CT, or 3D ultrasounds volume) reformatted images may be produced by interpolating from the sampling lattice with any appropriate standard interpolation scheme. If the desired data is two dimensional in nature (such as an Xray, or 2D ultrasound) the data may be projected onto the reformat plane, or its planar intersection only presented, or both approaches fused as desired. Once the reformatted planes are presented to the user, they may adjust the planar location within the 3D space, and refine the targeting position relative to each planar representation until they are satisfied that they have identified the correct location in 3D space.

Using the image or model of the 3D volume, the method includes designating a location of at least one incision into bone tissue.

Designation of the location of the one or more incision points may be done in one of several ways. For example, the clinician may select the incision point(s) by overlaying a mouse cursor on point(s) of the 3D rendered skull surface and clicking. Alternatively, the system may be programmed to automatically select or suggest potential incision point(s), based on certain criteria (such as the use of sulcal paths for entry to a brain target). For example, given an image volume (e.g. a T1 MRI image), a segmentation of that image including labeling of portions of the image (into white matter, grey matter, dura, and sulci), and a target, the system could be used to limit or suggest against certain entry locations. The system could generate the best sulcal entry points based on, for example, minimizing the number of impacted fibres, distance from the sulcal boundary to the target, and volume of white and/or grey matter displaced by the approach path. Such points could be found by exhaustive search or various standard methodologies (e.g. energy minimization). A simple approach could be advanced by utilizing additional information (more segmentation labels, biomechanical modeling, fluid dynamic modeling) to apply a more sophisticated analysis to the generation of the “best” candidate points. The surgeon would select from amongst these “best” candidate points, or could reject them and select one manually.

In another embodiment, surgical paths may be traced and recorded through use of a navigation system, while a clinician using tracked tools attempts different approaches toward a target in the 3D model that has been fabricated to model the actual patient's anatomy.

As illustrated in FIG. 7, the 3D model is used for surgical planning. The 3D model (2) is registered with the patient image data (15). Registration may be achieved using a tracked surgical tool (1), such as a pointer tool, to touch landmark anatomical features or fiduciary markers, or to trace an anatomical feature. Surgical incisions can be planned on the 3D model using the surgical pointer tool and traces registered with the patient data. The traces can be extruded from the skull surface to the surface of the skin to denote the incisions. Surgical incisions in the skull or cranium are made using tracked surgical tools to test and rehearse the surgical procedure on the model. The pre-surgical planning may further include removal of a target within the brain, such as a brain tumor resection. Following rehearsal of the surgical procedure, the bone flap is closed on the 3D model. Physiological characteristics such as soft tissue swelling or bleeding may be emulated by the 3D model to present the practitioner with potential surgical events and complications.

In another example, the practitioner may trace an incision in the bone and make the incision to remove part of the bone tissue of the 3D model. The planning may then include replacing the piece of removed bone, or making several incisions to remove bone fragments and replace the bone fragments to correct a patient-specific skull structure. Several attempts may be made in replacing the bone fragment(s), until the ideal placement and procedure is achieved. For pre-surgical planning of procedures such as pediatric surgeries, the bone can be deformed, re-shaped and/or flexed, or pieces can be cut out, until the desired result is obtained. In the case of a virtual 3D model, sections of virtual skull can be removed and reassembled in 3D space showing their position relative to each other. In some planning procedures, sections of bone can be deformed and flexed as required to increase the volume of the calvarium. Initial calvarium volume can be computed and optimal/target volumes can be described and set. Calvarium volume may include the shape of the calvarium. The procedures on the 3D model are recorded by the planning system using the tracked surgical tools. The practitioner may test and rehearse the procedure until the desired result is obtained, then the procedure can be ported from the planning software into the navigation system for use in surgery.

Surgical tools and the patient model may be physical in nature, or they may be virtual or a mixture of physical and virtual representations. The tracked surgical tools may be surgical tools as used in surgery or virtual tools overlaid on a hand-held device tracked by the planning system.

Implants for bone replacement or correction, such as for a craniofacial reconstruction, may be produced by 3D printing, using patient imaging data to determine the dimensions of the required implant. An alternate method of inputting data for planning an implant is to map the implant by tracing the dimensions of the required implant or patient anatomy using a pointer tool. For correction of bone structure that is deformed or injured on one side of a patient anatomy, the structure of the opposite side may be mapped or traced with a pointer tool, inversed and used to provide anatomical dimensions for an implant, for example by 3D printing. The implant can be tested in the pre-surgical planning system by performing a bone incision in the 3D model and inserting the implant to test the fit and placement of the implant with the patient-specific model.

For surgeries requiring more complex reconstruction, pre-surgical planning may include placement of the bone or implant and projecting the information back to a 3D scanner. More information related to the placement of bone may be found in U.S. Pat. No. 9,913,733 filed on Jul. 7, 2016, entitled “INTRA-OPERATIVE DETERMINATION OF DIMENSIONS FOR FABRICATION OF ARTIFICIAL BONE FLAP” wherein the contents are incorporated by reference in its entirety.

The surgical procedure can be iterated and recorded by the planning software, until the optimal steps are determined for obtaining the desired surgical result. For example, bone may be excised and then re-inserted with different arrangements until the correct placement is obtained. The recorded sequence can be ported from the planning software to the navigation system for intraoperative use in surgery.

Virtual anatomical models and surgical tools may be provided using a video camera and monitor for projection of data. The video camera may be a handheld device, such as a phone, or a wearable device such as the Microsoft® hollow lens. The phone can be held to capture the 3D model on the phone camera, so the image is updated with a screen that moves along with the camera.

Once the one or more incisions and bone placement paths have been produced they may be stored in the storage medium and visually displayed to the clinician. The one or more surgical intents may be selected by the surgeon checking off from a list of surgical outcome criteria displayed on the computer display screen by overlaying the mouse over the one or more listed intents and clicking them off. Further embodiments may include the use of a touch screen or a stylus, as well as a monitor in connection with a video tracking system or other means of delivering gestural input, or voice input. These surgical outcome criteria will be different for different parts of anatomy being worked on, for example the list of criteria may be different for case of brain surgery compared to spinal surgery.

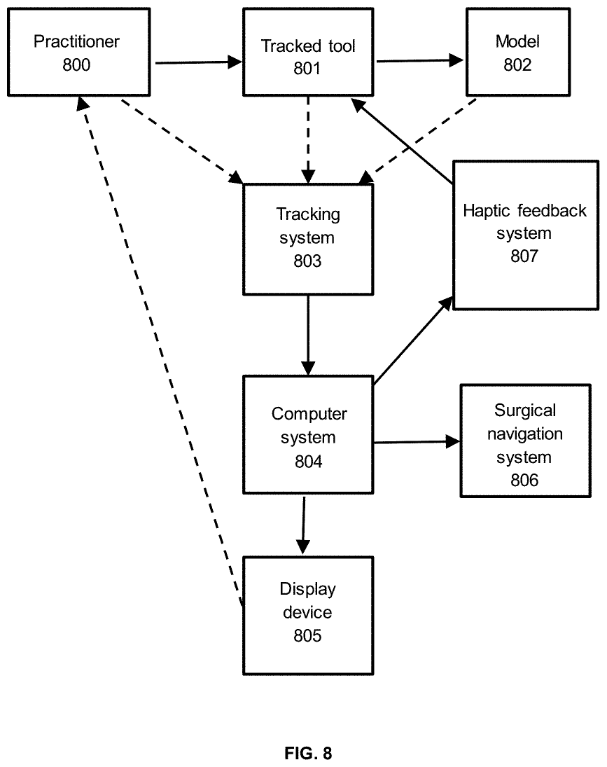

FIG. 8 depicts an embodiment of the surgical planning system. In FIG. 8, the dashed lines indicate the transmission and reception of light reflected from the practitioner 800, the tracked tool 801 and the model 802, as well as light generated by the display device 805 that is viewable by the practitioner 800. The solid lines indicate physical or electronic interaction.

The system is adapted to assist in testing, rehearsing and planning a surgical procedure on an anatomical region of a body. For example, the surgical procedure may involve performing a craniotomy and resecting a brain tumor in the brain of a human patient.

A model 802 of the patient's head and brain is employed in the case of a craniotomy and tumor resection. The model may include a physical feature representing a brain tumor inside the brain of a model 802, or the brain tumor to be resected may be partly or completely generated by the computer system. The practitioner 800 manipulates one or more tracked tools 801 to perform the surgical procedure. The tracked tool 801 may be a complete surgical instrument suitable for use in actual surgery, or it may be a partial instrument, for example not including a complete cutting tip.

The tracking system 803 tracks the position and orientation of the tracked tool 801 relative to the model 802 as the practitioner 800 manipulates the tracked tool 801 to perform the surgical procedure. In some embodiments, such as where the display device is a head-mounted display, the tracking system 803 also tracks the location of the practitioner 800, or more specifically the location of the practitioner's head and eyes relative to the model 802, and more specifically relative to the portion of the model upon which the surgical procedure is being performed. The tracking system 803 provides the position and orientation of the tracked tool 901 to the computer system 804 in real-time as the simulated surgical procedure is performed. Updated information may be provided by the tracking system 803 at regular intervals, for example, or may be provided when the tracking system 803 has detected a change in the location or orientation of a tracked tool 801. Such changes may result from actions by the practitioner such as repositioning the tracked tool 801, which may involve using the tracked tool 801 to cut and/or remove a portion of the model 802 or of the augmented physical model.

In addition to providing the above-described positional information, the tracking system 803 may provide images showing the model as the procedure progresses. For example, the tracking system 803 may include a video camera that provides a real-time stream of video showing the portion of the model 802 upon which the surgical procedure is being performed to the computer system 804. The model 802 will change, for example, as a trainee uses a surgical instrument to physically remove portions of the model 802, such as physical tumors disposed therein.

The computer system 804 receives the tracking system information and, based on that information, generates and displays on the display device 805 a display showing the model and a depiction of the tracked tool 801, which may be an augmented tracked tool, as discussed below. The practitioner 800 views the display on the display device 805 and manipulates the tracked tool 801 while viewing the display in order to perform the surgical procedure. The display device 805 may be a monitor at some distance from the practitioner 800, such as 1-5 meters, or it may be a head-mounted display that moves as the practitioner's head moves during the procedure.

The model 802 is generated by the computer system 805 based on images of the physical model upon which the surgical procedure is being performed with virtual elements rendered by the computer system and superimposed on a digital rendering of the physical model derived from the video images received from the tracking system 803. The extent and nature of the virtual elements varies substantially between different embodiments. In some embodiments, for example, the model 802 may include a simulated tumor and neural tracts, whereas in other embodiments some or all of the tumor and neural tracts may be generated by the computer system 804 as virtual elements that are superimposed on a rendering of the physical model by the computer system 804. In some embodiments, the virtual elements include arteries and if an artery is cut or damaged, then the virtual elements may include blood exiting the cut or damaged artery.

In some embodiments, the virtual elements may include visual highlighting of certain portions of the model to provide information to the practitioner 800.

Preferably, the display is regenerated and displayed in real-time as the practitioner 800 manipulates the tracked tools 801 and modifies the model 802 or the augmented physical model by modifying virtual elements by manipulating the tracked tool 801. This may be done by continuously augmenting a received video stream of the physical model as it is received and adding virtual elements. The added virtual elements generally change in accordance with the practitioner's manipulation of the tracked tool 801 as the model 802 is modified, for example thereby exposing different internal structures that require additional virtual elements. They also change in accordance with the simulated impact of the tracked tool 801 on the displayed virtual elements.

In all embodiments, the computer system 804 stores the simulated surgical procedure in memory by storing the information from the tracking system, and the procedure that provides the desired result may be saved and used for intra-operative surgical navigation, as detailed below.

In some embodiments, the tracked tool 801 may not be a complete surgical instrument but rather may resemble a portion of a surgical instrument and not include all portions of the surgical instrument. For example, a cutting tip may not be present in the tracked tool 801. In such embodiments, portions of the surgical instrument not present in the tracked tool 801 may be generated as a virtual element or elements rendered by the computer system 804 as part of an augmented tracked tool in the generated display. For example, a virtual cutting tip on an instrument in the generated display may be manipulated by the practitioner 800 by manipulating the tracked tool 801 to resect a tumor generated as a virtual element of the augmented physical model. In such embodiments, since there is little or no physical contact between the tracked tool 801 and the model 802, the system may include a haptic feedback mechanism 807 coupled to the tracked tool 801 and configured to provide haptic feedback to the practitioner 800 under control of the computer system 804 by applying force to the tracked tool 801 as the practitioner 800 moves the tracked tool 801. The magnitude and direction of the force is calculated by the computer system 805 to approximate what would be experienced by a corresponding surgical instrument in a real operation where the surgical instrument is interacting with the patient's body in a manner corresponding to the interaction of the tracked tool, in the case of a complete tracked tool, or otherwise an augmented tracked tool, with the model 802. In the case of an augmented tracked tool where the cutting tip is virtual, then most of the haptic feedback would need to be provided by the haptic feedback mechanism 807. In a situation where a full tracked tool is used to perform a procedure involving primarily physical elements of the model 802, then the haptic feedback mechanism 807 is generally not required as the appropriate forces are created by the interaction of the tracked tool 801 with the model 802.

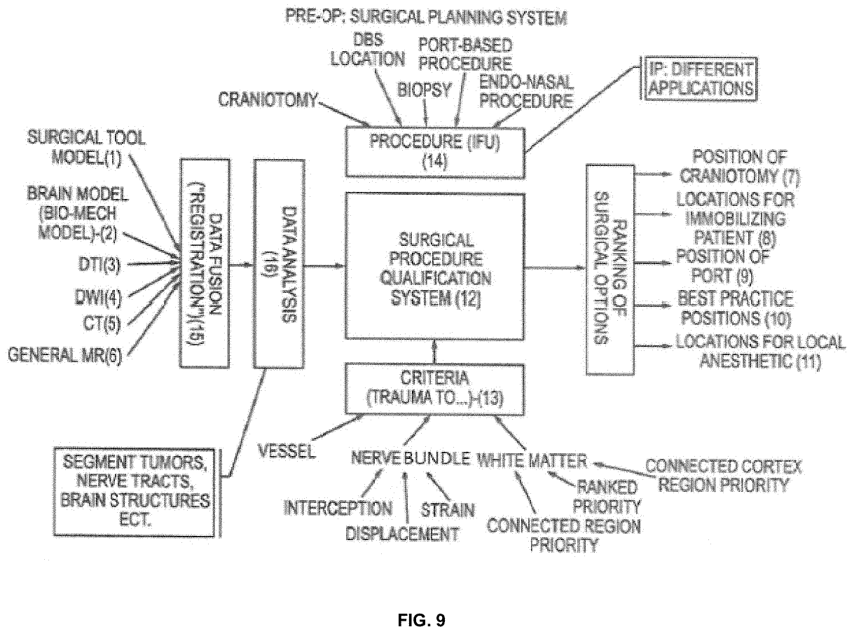

FIG. 9 shows an embodiment of the present method and system, for use as an intra-operative multi-modal surgical planning and navigation system and method. The system and method can be used as a surgical planning and navigation tool in the pre-operative and intra-operative stages. Persons of skill will appreciate that the data input(s) of the surgical planning steps and surgical procedures described in FIGS. 7 and 8 can be used as input(s) to the intra-operative navigation stage described in FIG. 9, through the use of patient fiducial markers visible in the imaging, or other imaging techniques, examples of which are known in the art.

The embodiment of FIG. 9 provides a user, such as a surgeon, with a unified means of navigating through a surgical region by utilizing pre-operative data input(s) and updated intra-operative data input(s). The processor(s) of system and methods are programmed with instructions/algorithms 11 to analyze pre-operative data input(s) and intraoperative data input(s) to update surgical plans during the course of surgery. For example, if intra-operative input(s) in the form of newly acquired images identified a previously unknown nerve bundle or brain tract, these input(s) can, if desired, be used to update the surgical plan during surgery to avoid contacting the nerve bundle. Persons of skill will appreciate that intraoperative input(s) may include a variety input(s) including local data gathered using a variety of sensor(s).

In some embodiments, the system and methods of FIG. 9 may provide continuously updated intra-operative input(s) in the context of a specific surgical procedure by means of intraoperative imaging sensor(s) to validate tissue position, update tissue imaging after tumor resection and update surgical device position during surgery.

The systems and methods may provide for re-formatting of the image, for example, to warn of possible puncture of critical structures with the surgical tools during surgery, or collision with the surgical tool during surgery. In addition, the embodiments disclosed herein may provide imaging and input updates for any shifts that might occur due to needle deflection, tissue deflection or patient movement as well as algorithmic approaches to correct for known imaging distortions. The magnitude of these combined errors is clinically significant and may regularly exceed 2 cm. Some the most significant are MRI based distortions such gradient non-linearity, susceptibility shifts, eddy current artifacts which may exceed 1 cm on standard MRI scanners (1.5T and 3.0T systems).

Persons of skill will appreciate that a variety of intraoperative imaging techniques can be implemented to generate intra-operative input(s) including anatomy specific MRI devices, surface array MRI scans, endo-nasal MRI devices, anatomy specific US scans, endo-nasal US scans, anatomy specific CT or PET scans, port-based or probe based photo-acoustic imaging, as well as optical imaging done with remote scanning, or probe based scanning.

Persons of skill will appreciate that a variety of intraoperative imaging techniques can be implemented to generate intra-operative input(s) including anatomy specific MRI devices, surface array MRI scans, endo-nasal MRI devices, anatomy specific US scans, endo-nasal US scans, anatomy specific CT or PET scans, port-based or probe based photo-acoustic imaging, as well as optical imaging done with remote scanning, or probe based scanning.

Generally, a computer, computer system, computing device, client or server, as will be well understood by a person skilled in the art, includes one or more than one electronic computer processor, and may include separate memory, and one or more input and/or output (I/O) devices (or peripherals) that are in electronic communication with the one or more processor(s). The electronic communication may be facilitated by, for example, one or more busses, or other wired or wireless connections. In the case of multiple processors, the processors may be tightly coupled, e.g. by high-speed busses, or loosely coupled, e.g. by being connected by a wide-area network.

A computer processor, or just “processor”, is a hardware device for performing digital computations. It is the express intent of the inventors that a “processor” does not include a human; rather it is limited to be an electronic device, or devices, that perform digital computations. A programmable processor is adapted to execute software, which is typically stored in a computer-readable memory. Processors are generally semiconductor based microprocessors, in the form of microchips or chip sets. Processors may alternatively be completely implemented in hardware, with hard-wired functionality, or in a hybrid device, such as field-programmable gate arrays or programmable logic arrays. Processors may be general-purpose or special-purpose off-the-shelf commercial products, or customized application-specific integrated circuits (ASICs). Unless otherwise stated, or required in the context, any reference to software running on a programmable processor shall be understood to include purpose-built hardware that implements all the stated software functions completely in hardware.

Multiple computers (also referred to as computer systems, computing devices, clients and servers) may be networked via a computer network, which may also be referred to as an electronic network or an electronic communications network. When they are relatively close together the network may be a local area network (LAN), for example, using Ethernet. When they are remotely located, the network may be a wide area network (WAN), such as the internet, that computers may connect to via a modem, or they may connect to through a LAN that they are directly connected to.

Computer-readable memory, which may also be referred to as a computer-readable medium or a computer-readable storage medium, which terms have identical (equivalent) meanings herein, can include any one or a combination of non-transitory, tangible memory elements, such as random access memory (RAM), which may be DRAM, SRAM, SDRAM, etc., and nonvolatile memory elements, such as a ROM, PROM, FPROM, OTP NVM, EPROM, EEPROM, hard disk drive, solid state disk, magnetic tape, CDROM, DVD, etc.) Memory may employ electronic, magnetic, optical, and/or other technologies, but excludes transitory propagating signals so that all references to computer-readable memory exclude transitory propagating signals. Memory may be distributed such that at least two components are remote from one another, but are still all accessible by one or more processors. A nonvolatile computer-readable memory refers to a computer-readable memory (and equivalent terms) that can retain information stored in the memory when it is not powered. A computer-readable memory is a physical, tangible object that is a composition of matter. The storage of data, which may be computer instructions, or software, in a computer-readable memory physically transforms that computer-readable memory by physically modifying it to store the data or software that can later be read and used to cause a processor to perform the functions specified by the software or to otherwise make the data available for use by the processor. In the case of software, the executable instructions are thereby tangibly embodied on the computer-readable memory. It is the express intent of the inventor that in any claim to a computer-readable memory, the computer-readable memory, being a physical object that has been transformed to record the elements recited as being stored thereon, is an essential element of the claim.

Software may include one or more separate computer programs configured to provide a sequence, or a plurality of sequences, of instructions to one or more processors to cause the processors to perform computations, control other devices, receive input, send output, etc.

It is intended that the invention includes computer-readable memory containing any or all of the software described herein. In particular, the invention includes such software stored on nonvolatile computer-readable memory that may be used to distribute or sell embodiments of the invention or parts thereof.

While the Applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

What is claimed is:

1. A planning system for a surgical procedure by a practitioner on a patient anatomical region, comprising:
a model of the patient anatomical region, wherein the model includes a simulated bone;
a tracked tool for interacting with the model;
a tracking system configured to track a tracked tool location and orientation relative to the model;
a display device for viewing the model and the tracked tool; and
a computer system electronically connected to the tracking system and the display device, the computer system being configured to:
receive information from the tracking system indicating the tracked tool location and orientation;
generate and display on the display device a display comprising the model and the tracked tool, wherein the display is updated based on changes in the tracked tool location and orientation relative to the model as the tracked tool performs the surgical procedure on the model; and
store the tracking system information in a computer readable memory, wherein the tracking system information is portable to a surgical navigation system.

2. The planning system of claim 1, wherein the model is a replica of the patient anatomical region.

3. The planning system of claim 1, wherein the model comprises a physical model.

4. The planning system of claim 3, wherein the generate and display step comprises virtual elements rendered by the computer system and superimposed on a digital rendering of the physical model.

5. The planning system of claim 1, wherein the tracking system comprises a video camera that provides a real-time stream of video showing a portion of the model upon which the surgical procedure is being performed, and wherein the computer system generates and updates the model based on the video camera.

6. The planning system of claim 1, wherein the anatomical region comprises a portion of a skull having a bone deformation and the surgical procedure comprises restructuring the skull to correct the bone deformation.

7. The planning system of claim 1, wherein the display device is a head-mounted display worn by the practitioner.

8. The planning system of claim 7, wherein the tracking system is further configured to track a location of the practitioner's head relative to the model, wherein the display is generated from a perspective of the practitioner based on the location of the practitioner's head, and wherein the display is updated as the location of the practitioner's head changes.

9. The planning system of claim 1, wherein the computer system is further configured to generate and display on the display device an augmented tracked tool, wherein a location and orientation of the augmented tracked tool relative to the model as depicted in an image are based on the location and orientation of the tracked tool relative to the model, and wherein a displayed augmented tracked tool is updated on the display device by the computer system as the practitioner manipulates the tracked tool.

10. The planning system of claim 9, wherein the tracked tool resembles a portion of a surgical instrument, wherein the augmented tracked tool comprises a digital rendering of the tracked tool augmented by a tip portion generated by the computer system so that the augmented tracked tool appears on the display device to be the surgical instrument.

11. The planning system of claim 9 further including a haptic feedback mechanism coupled to the tracked tool and configured to provide haptic feedback to the practitioner under control of the computer system by applying force to the tracked tool as the practitioner moves the tracked tool.

12. A method of surgical planning, comprising:
acquiring a volumetric data of a patient anatomical region using a medical imaging methodology;
extracting a three-dimensional model of the patient anatomical region from the volumetric data;
providing the model;
registering the model with the volumetric data;
using a tracked tool to perform a simulated surgical procedure on the model, wherein the simulated surgical procedure comprises removing a bone section from the model and reassembling the model with an implant;
viewing the model and the tracked tool on a display device;
recording the simulated surgical procedure with a tracking system;
storing the simulated surgical procedure in a computer readable memory; and
porting the simulated surgical procedure into a surgical navigation system.

13. The method of claim 12, wherein providing the model comprises at least one of printing a three-dimensional model and providing a computer-generated virtual image.

14. The method of claim 13, further comprising superimposing the virtual image on a digital rendering of the printed model on the display device.

15. The method of claim 12, wherein using the tracked tool comprises at least one of: tracking entry points; tracking incision points; and tracking closure systems.

16. The method of claim 15, wherein tracking closure systems comprises tracing a position of the implant in the model using the tracked tool.

17. The method of claim 12, wherein reassembling the model with the implant comprises re-inserting the bone section.

18. The method of claim 12, wherein reassembling the model with the implant comprises deforming and flexing the implant.

19. The method of claim 12, further comprising:
calculating a target post-surgical volume corresponding to a target model volume after reassembling the model with the implant; and
placing the implant to provide the target post-surgical volume.

20. The method of claim 12, wherein using the tracked tool on the model further comprises mapping the implant to replicate a model anatomical detail.
